(19) United States
(12) Patent Application Publication
(10) Pub. No.: US 2007/0026065 A1
(43) Pub. Date: Feb. 1, 2007

(54) Solid, modified-release pharmaceutical dosage forms which can be administered orally

(75) Inventors: Klaus Benke, Bergisch Gladbach (DE); Jan-Olav Henck, West Lafayette, IN (US)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 11/317,720

(22) Filed: Dec. 23, 2005

(30) Foreign Application Priority Data
Dec. 24, 2004 (DE) 102004062475.5

(51) Int. Cl. A61K 31/5377; A61K 9/22

(52) U.S. Cl. 424/468; 514/235.2

(57) ABSTRACT

The present invention relates to solid, modified-release pharmaceutical dosage forms which can be administered orally and comprise 5-chloro-N-({(5S)-2-oxo-3-[4-(3-oxo-4-morpholinyl)phenyl]-1,3-oxazolidin-5-yl}methyl)-2-thiophenecarboxamide, and process for their production, their use as medicaments, their use for the prophylaxis, secondary prophylaxis and/or treatment of disorders, and their use for producing a medicament for the prophylaxis, secondary prophylaxis and/or treatment of disorders.

The present invention relates to solid, modified-release pharmaceutical dosage forms which can be administered orally and comprise 5-chloro-N-({(5S)-2-oxo-3-[4-(3-oxo-4-morpholinyl) phenyl]-1,3-oxazolidin-5-yl}methyl)-2-thiophenecarboxamide, and process for their production, their use as medicaments, their use for the prophylaxis, secondary prophylaxis and/or treatment of disorders, and their use for producing a medicament for the prophylaxis, secondary prophylaxis and/or treatment of disorders.

Modified-release dosage forms mean according to the invention preparations whose active ingredient release characteristics after intake are adjusted in relation to time, profile and/or site in the gastrointestinal tract in a way which cannot be achieved after administration of conventional formulations (e.g. oral solutions or solid dosage forms which release active ingredient rapidly, alternative terms are frequently also used, such as “slow release”, “delayed”). Besides the term “modified release” or “controlled release”. These are likewise encompassed by the scope of the present invention.

Various methods are known for producing modified-release pharmaceutical dosage forms, see, for example, B. Lippold in “Oral Controlled Release Products: Therapeutic and Biopharmaceutic Assessment” edited by U. Gundert-Remy and H. Möller, Stuttgart, Wiss. Verl.-Ges., 1989, 39-57.

5-Chloro-N-({(5S)-2-oxo-3-[4-(3-oxo-4-morpholinyl)phenyl]-1,3-oxazolidin-5-yl}methyl)-2-thiophenecarboxamide (I) is a low molecular weight inhibitor of coagulation factor Xa which can be administered orally and can be employed for the prophylaxis, secondary prophylaxis and/or treatment of various thromboembolic disorders (concerning this, see WO-A 01/47919, the disclosure of which is incorporated herein by reference). When active ingredient (I) is mentioned hereinafter, this encompasses all crystal modifications and the amorphous form of 5-chloro-N-({(5S)-2-oxo-3-[4-(3-oxo-4-morpholinyl)phenyl]-1,3-oxazolidin-5-yl}methyl)-2-thiophenecarboxamide (I) and the respective hydrates, solvates and cocrystals.

For the diseases requiring treatment over a lengthy period, or for the long-term prophylaxis of diseases, it is desirable to minimize the frequency of intake of medicaments. This is not only more convenient for the patient, but also increases the treatment reliability (compliance) by reducing the disadvantages of irregular intakes. The desired reduction in the frequency of intake, for example from twice a day to once a day administration, can be achieved by prolonging the therapeutically effective plasma levels through modified release of active ingredient from the dosage forms.

After intake of dosage forms with modified release of active ingredient, it is additionally possible to diminish the occurrence of unwanted side effects correlated with peak concentrations by smoothing the plasma profile (minimizing the so-called peak to trough ratio), that is to say by avoiding high plasma concentrations of active ingredient, which are frequently observed after administration of fast-release pharmaceutical forms.

It is advantageous, especially for the long-term therapy or prophylaxis and secondary prophylaxis of arterial and/or venous thromboembolic disorders (for example deep vein thromboses, stroke, myocardial infarction and pulmonary embolism), to have the active ingredient (I) available in a form which, through a modified release of active ingredient, leads to a reduction in the peak to trough ratio and makes once a day administration possible.

It is additionally necessary in the development of formulations to take account of the physicochemical and biological properties of the active ingredient (I), for example the relatively low solubility in water (about 7 mg/l; 25° C.), the relatively high melting point of about 230° C. of the active ingredient (I) in the crystal modification in which the active ingredient (I) is obtained when prepared by the route described in Example 44 of WO 01/47919 and which is referred to hereinafter as modification I, and the plasma half-life of about 7 hours. Accordingly, for the desired once a day administration, specific pharmaceutical formulations with modified release of the active ingredient (I), taking account of its physicochemical and biological properties, are required.

DE 10355461 describes pharmaceutical dosage forms which comprise the active ingredient (I) in hydrophylized form. Preferred in this connection are fast-release tablets which have a Q value (30 minutes) of 75% in the USP release method with apparatus 2 (paddle).

It has now surprisingly been found that dosage forms which release the active ingredient (I) at a particular, defined modified rate make once a day administration possible with comparatively constant plasma concentrations.

The present invention relates to solid, modified-release pharmaceutical dosage forms which can be administered orally and comprise 5-chloro-N-({(5S)-2-oxo-3-[4-(3-oxo-4-morpholinyl) phenyl]-1,3-oxazolidin-5-yl}methyl)-2-thiophenecarboxamide (I), characterized in that 80% of the active ingredient (I) (based on the stated total amount of the active ingredient) are released over a period of from at least 2 and at most 24 hours in the USP release method with apparatus 2 (paddle).

In a preferred embodiment of the present invention, 80% of the active ingredient (I) are released in a period of from 4 to 20 hours in the USP release method with apparatus 2 (paddle).

The active ingredient (I) may be present in the pharmaceutical dosage forms of the invention in crystalline form or in noncrystalline amorphous form or in mixtures of crystalline and amorphous active ingredient fractions.

If the dosage forms of the invention comprise the active ingredient (I) in crystalline form, in a preferred embodiment of the present invention the active ingredient (I) is employed in micronized form of crystal modification 1. In this case, the active ingredient (I) preferably has an average particle size X₅₀ of less than 10 μm, in particular of less than 8 μm, and an X₉₀ value (90% fraction) of less than 20 μm, in particular of less than 15 μm.

In a further preferred embodiment of the present invention, when crystalline active ingredient (I) is used the micronized active ingredient (1) is present in hydrophylized form, thus increasing its rate of dissolution. The preparation of hydrophylized active ingredient (I) is described in detail in DE 10355461, the disclosure of which is incorporated herein by reference.

The active ingredient (I) is, however, preferably present in the pharmaceutical dosage forms of the invention not in crystalline form but completely or predominantly in amorphous form. A great advantage of the amorphisation of the active ingredient is the increase in the solubility of active ingredient and thus the possibility of increasing the fraction of active ingredient (I) absorbed, in particular from lower sections of the intestine.

Various pharmaceutically suitable production methods are conceivable for amorphisation of the active ingredient (I).

In this connection, the dissolving method in which an active ingredient and excipient(s) employed where appropriate are dissolved and then further processed is less suitable because the crystalline active ingredient (I) has only a limited solubility in pharmaceutically suitable organic solvents such as, for example, acetone or ethanol, and therefore disproportionately large amounts of solvent must be used.

The method preferred according to the invention for amorphisation of the active ingredient (I) is the melting method in which an active ingredient is melted together with one or more suitable excipients.

Particular preference is given in this connection to the melt extrusion method [Breitenbach, J., “Melt extrusion: from process to drug delivery technology”, European Journal of Pharmaceutics and Biopharmaceutics 54 (2002), 107-117; Breitenbach, J., “Feste Lösungen durch Schmelzextrusion—ein integriertes Herstellkonzept”, Pharmazie in unserer Zeit 29 (2000), 46-49].

It can be ensured in this method, through choice of a suitable formulation and suitable production parameters, that the degradation of active ingredient does not exceed pharmaceutically acceptable limits. This is a difficult task with a melting point of about 230° C. for the active ingredient (I) in crystal modification I, because significant rates of decomposition of the active ingredient and/or of the excipients are usually to be expected in this high temperature range.

The melt extrusion method for preparing the active ingredient (I) in amorphous form is carried out according to the invention in the presence of a polymer such as, for example, polyvinylpyrrolidones, polyethylene glycols (PEG), polymethacrylates, polymethylmethacrylates, polyethylene oxides (especially water-soluble polyethylene oxide resins such as, for example, POLYOX™ Water Soluble Resins, Dow), polyoxyethylene-polyoxypropylene block copolymers, vinylpyrrolidone-vinyl acetate copolymers or of a cellulose ether such as, for example, hydroxypropylcellulose (HPC) or of a mixture of various polymers such as, for example, mixtures of two or more of the polymers mentioned. The preferred polymer in this connection is hydroxypropylcellulose (HPC), polyvinylpyrrolidone (PVP) or a mixture of HPC and PVP. The polymer in this connection is particularly preferably hydroxypropylcellulose (HPC) or polyvinylpyrrolidone (PVP).

The proportion of polymer in the melt extrudate is preferably according to the invention at least 50% of the total mass of the melt extrudate.

The active ingredient (I) is preferably present according to the invention in the melt extrudate in a concentration of between 1 and 20% based on the total mass of the melt extrudate.

It has proved advantageous in the melt extrusion method for preparing the active ingredient (I) in amorphous form to add one or more pharmaceutically suitable substances to depress the melting point of the active ingredient (I) or as plasticizers in order to reduce the degradation of active ingredient taking place during the extrusion process, and to facilitate processing.

These pharmaceutically suitable substances are preferably added according to the invention in a concentration of from 2 to 40% based on the total mass of the melt extrudate.

Examples suitable for this purpose are urea, polymers such as polyvinylpyrrolidones, polyethylene glycols, polymethacrylates, polymethylmethacrylates, polyoxyethylene-polyoxypropylene block copolymers, vinylpyrrolidone-vinylacetate copolymers or sugar alcohols such as, for example, erythritol, maltitol, mannitol, sorbitol and xylitol. Sugar alcohols are preferably employed. It must be ensured in this connection, by choice of suitable preparation parameters, that the active ingredient (I) is converted as completely as possible into the amorphous state, in order to increase the solubility of the active ingredient.

The extrudate comprising the active ingredient (I) and obtained by melt extrusion methods is cut, where appropriate rounded and/or coated and may for example be further processed to a sachet formulation or packed into capsules (multiple-unit formulations). A further possibility is for the extrudate obtained after melt extrusion to be mixed, after the cutting and grinding, with usual tabletting excipients, and compressed to tablets, and for the latter also to be coated subsequently where appropriate (single-unit formulations).

Various pharmaceutical oral dosage forms with modified release of active ingredient (I) can be employed according to the invention. Without restricting the scope of the present invention, preferred examples which may be mentioned thereof are:
1. tablet formulations (single units) based on erosion matrix systems
2. multiparticulate dosage forms with erosion—and/or diffusion—controlled release kinetics such as granules, pellets, mini tablets and pharmaceutical forms produced therefrom, such as, for example, sachets, capsules or tablets
3. dosage forms based on osmotic release systems
   1. Tablet Formulations Based On Erosion Matrix Systems

In this case, the modified release of active ingredient takes place through formulation of the active ingredient in an erodable matrix composed of one or more soluble polymers, with the release of active ingredient being dependent on the rate of swelling and dissolution or erosion of the matrix and on the rate of dissolution, solubility and rate of diffusion of the active ingredient. This principle for modified release of active ingredient is also known by the terms erosion matrix or hydrocolloid matrix system. The erosion/hydrocolloid matrix principle for modifying the release of active ingredient from pharmaceutical dosage forms is described for example in:
Alderman, D. A., “A review of cellulose ethers in hydrophilic matrixes for oral controlled-release dosage forms”, Int. J. Pharm. Tech. Prod. Mfr. 5 (1984), 1-9.
Melia, C. D., “Hydrophilic matrix sustained release systems based on polysaccharide carriers”, Critical Reviews in Therapeutic Drug Carrier Systems 8 (1991), 395-421.
Vazques, M. J. et al., “Influence of technological variables on release of drugs from hydrophilic matrices”, Drug Dev. Ind. Pharm. 18 (1992), 1355-1375.

The desired release kinetics can be controlled for example via the polymer type, the polymer viscosity, the polymer and/or active ingredient particle size, the active ingredient-polymer ratio and additions of further pharmaceutically usual excipients such as, for example, soluble or/and insoluble fillers.

Matrix formers suitable for the purposes of the present invention are numerous polymers, for example polysaccharides and cellulose ethers such as methylcellulose, carboxymethylcellulose, hydroxyethylmethylcellulose, ethylhydroxyethylcellulose, hydroxyethylcellulose, with hydroxypropylcellulose (HPC) or hydroxypropylmethylcellulose (HPMC) or mixtures of hydroxypropylcellulose and hydroxypropylmethylcellulose preferably being employed.

The matrix former is preferably present in the tablet formulations of the invention based on erosion matrix systems in a concentration of between 10 and 95% based on the total mass of the tablet.

The active ingredient (I) is preferably present in the tablet formulations of the invention based on erosion matrix systems in a concentration of between 1 and 50% based on the total mass of the tablet.

Besides the polymer(s) for forming the erosion (hydrocolloid) matrix and the active ingredient, it is possible to add to the tablet formulations further tabletting excipients familiar to the skilled person (e.g. binders, fillers, lubricants/glidants/flow aids). The tablets may additionally be covered with a coating.

Suitable materials for a photoprotective and/or coloured coating are for example polymers such as polyvinyl alcohol, hydroxypropylcellulose and/or hydroxypropylmethylcellulose, where appropriate in combination with suitable plasticizers such as, for example, polyethylene glycol or polypropylene glycol and pigments such as, for example, titanium dioxide or iron oxides.

Further examples of suitable materials for producing a coating are aqueous dispersions such as, for example, ethylcellulose dispersion (e.g. Aquacoat, FMC) or poly(ethyl acrylate, methyl methacrylate) dispersion (Eudragit NE 30 D, Röhm/Degussa). It is also possible to add plasticizers and wetting agents to the coating (e.g. triethyl citrate or polysorbates), non-stick agents such as, for example, talc or magnesium stearate and hydrophilic pore formers such as, for example, hydroxypropylmethylcellulose, polyvinylpyrrolidone or sugar. The coating substantially has the effect that a delay in release of the active ingredient is possible for the first one to a maximum of two hours after administration.

Further materials suitable for producing a coating are substances to achieve a resistance to gastric juice, such as, for example, anionic polymers based on methacrylic acid (Eudragit L+S, Röhm/Degussa) or cellulose acetate phthalate.

Methods suitable for producing tablet formulations of the invention comprising the active ingredient (I) in crystalline or predominantly crystalline form are the usual ones known to the skilled person, such as direct tabletting, tabletting after dry granulation, melt granulation, extrusion or wet granulation such as, for example, fluidized bed granulation.

However, the active ingredient (I) is preferably employed in amorphous or predominantly amorphous form, in particular as melt extrudate, for the tablet formulations of the invention based on erosion matrix systems, so that the active ingredient (I) is present in the finished formulation in amorphous form.

The present invention further relates to a process for producing the tablet formulation of the invention based on erosion matrix systems, where an extrudate comprising the active ingredient (I) is produced, preferably with the aid of melt extrusion, and is then ground, mixed with further tabletting excipients known to the skilled person (matrix formers, binders, fillers, lubricants/glidants/flow aids) and then compressed, preferably by direct tabletting, to tablets which may finally be covered with a coating.

2. Multiparticulate Dosage Forms such as Granules, Pellets, Mini Tablets, and Capsules, Sachets and Tablets Produced therefrom

Besides the so-called “single unit” also suitable for active ingredient (I) are multiparticulate dosage forms whose modified release of active ingredient takes place under erosion/diffusion control. The term “multiparticulate dosage forms” means according to the invention those formulations which, in contrast to single units (tablets), consist of a plurality of small particles such as granular particles, spherical granules (pellets) or mini tablets. The diameter of these particles is ordinarily between 0.5 and 3.0 mm, preferably between 1.0 and 2.5 mm.

The advantage of these multiparticulate systems by comparison with single units is that the intra- and interindividual variability of gastrointestinal passage is usually smaller, resulting in a smaller variability of the plasma profiles and often also reduced dependence on food (food effect), i.e. diminished differences after administration on a full or empty stomach. The granules (pellets) or small-format tablets (mini tablets with diameter not exceeding 3 mm) can be packed into capsules or be prepared as sachet. A further possibility is further processing to larger tablets which, after contact with water/gastric juice, release the primary granules/pellets by rapid disintegration.

The excipients and processes suitable for producing multiparticulate pharmaceutical dosage forms comprising the active ingredient (I) are in principle all those mentioned in section 1.

The matrix former employed in this case is preferably a polymer from the group of cellulose ethers, in particular hydroxypropylcellulose (HPC) or hydroxypropylmethylcellulose (HPMC) or a mixture of hydroxypropylcellulose and hydroxypropylmethylcellulose.

The polymer is preferably present in the pharmaceutical dosage forms of the invention based on multiparticulate dosage forms in a concentration of between 10 and 99%, in particular between 25 and 95%, based on the total mass of the composition.

The active ingredient (I) is preferably present in the pharmaceutical dosage forms of the invention based on multiparticulate dosage forms in a concentration of between 1 and 30% based on the total mass of the composition.

The extrusion/spheronization process which is described for example in Gandhi, R., Kaul, C. L., Panchagnula, R., “Extrusion and spheronization in the development of oral controlled-release dosage forms”, Pharmaceutical Science & Technology Today Vol. 2, No. 4 (1999), 160-170, is particularly suitable for producing pellets which comprise the active ingredient (I) in crystalline or predominantly crystalline form.

In a preferred embodiment of the present invention, the multiparticulate dosage forms comprise the active ingredient (I) in amorphous form and are moreover produced preferably by the melt extrusion method.

The particles/pellets/mini tablets may be coated where appropriate, for example with aqueous dispersions such as, for example, ethylcellulose dispersion (e.g. Aquacoat, FMC) or a poly(ethyl acrylate, methyl methacrylate) dispersion (Eudragit NE 30 D, Röhm/Degussa). It is also possible to add plasticizers and wetting agents to the coating (e.g. triethyl citrate or polysorbates), non-stick agents such as, for example, talc or magnesium stearate and hydrophilic pore formers such as, for example, hydroxypropylmethylcellulose, polyvinylpyrrolidone or sugar. The coating substantially has the effect that a delay in release of the active ingredient is possible for the first one to a maximum of two hours after administration.

Further materials suitable for producing a coating are substances to achieve a resistance to gastric juice, such as, for example, anionic polymers based on methacrylic acid (Eudragit L+S, Röhm/Degussa) or cellulose acetate phthalate.

The present invention further relates to pharmaceutical dosage forms, preferably capsules, sachets or tablets, comprising the multiparticulate dosage forms described above.

The present invention further relates to a process for producing the multiparticulate pharmaceutical dosage forms of the invention, where an extrudate comprising the active ingredient (I) in amorphous form is obtained preferably by melt extrusion. In a preferred embodiment of the present invention, a multiparticulate dosage form in pellet form is produced directly by cutting this extrudate strand and, where appropriate, subsequent rounding. The pellets obtained in this way can then be covered with a coating and be packed into capsules or a sachet.

3. Osmotic Release Systems

Further suitable dosage forms with modified release of the active ingredient (I) are based on osmotic release systems. In these cases, cores, for example capsules or tablets, preferably tablets, are enveloped by a semipermeable membrane which has at least one orifice. The water-permeable membrane is impermeable to the components of the core but permits water to enter the system from outside by osmosis. The water which penetrates in then releases, through the osmotic pressure produced, the active ingredient in dissolved or suspended form from the orifice(s) in the membrane. The total active ingredient release and the release rate can substantially be controlled via the thickness and porosity of the semipermeable membrane, the composition of the core and the number and size of the orifice(s). Advantages, formulation aspects, use forms and information on production processes are described inter alia in the following publications:
Santus, G., Baker, R. W., “Osmotic drug delivery: a review of the patent literature”, Journal of Controlled Release 35 (1995), 1-21
Verma, R. K., Mishra, B., Garg, S., “Osmotically controlled oral drug delivery”, Drug Development and Industrial Pharmacy 26 (7), 695-708 (2000)
Verma, R. K., Krishna, D. M., Garg, S., “Formulation aspects in the development of osmotically controlled oral drug delivery systems”, Journal of Controlled Release 79 (2002), 7-27
U.S. Pat. Nos. 4,327,725, 4,765,989, US 20030161882, EP 1 024 793.

Both single-chamber systems (elementary osmotic pump) and two-chamber systems (push-pull systems) are suitable for the active ingredient (I). The active ingredient (I) may be present in the osmotic systems both in crystalline, preferably micronized form, and in amorphous form or in mixtures with crystalline and amorphous fractions.

The shell of the osmotic pharmaceutical release system consists in both the single-chamber system and in the two-chamber system of a water-permeable material which is impermeable for the components of the core. Such shell materials are known in principle and described for example in EP-B1-1 024 793, pages 3-4, the disclosure of which is incorporated herein by reference. Preferably employed as shell material according to the invention are cellulose acetate or mixtures of cellulose acetate and polyethylene glycol.

A coating, for example a photoprotective and/or coloured coating, can be applied to the shell if required. Materials suitable for this purpose are for example polymers such as polyvinyl alcohol, hydroxypropylcellulose and/or hydroxypropylmethylcellulose, where appropriate in combination with suitable plasticizers such as, for example, polyethylene glycol or polypropylene glycol and pigments such as, for example, titanium dioxide or iron oxides.

The core in the osmotic single-chamber system preferably comprises:
2 to 30% active ingredient (I)
20 to 50% xanthan,
10 to 30% of a vinylpyrrolidone-vinyl acetate copolymer,
   where the difference from 100% is formed where appropriate by one or more additional ingredients selected from the group of further hydrophilic, swellable polymers, osmotically active additives and pharmaceutically usual excipients. The total of the core ingredients amounts to 100%, and the % data are based in each case on the total mass of the core.

The osmotic single-chamber system comprises as one of the essential ingredients of the core the hydrophilic water-swellable polymer xanthan. This is an anionic heteropolysaccharide which is obtainable commercially for example under the name Rhodigel® (produced by Rhodia). It is present in an amount of from 20 to 50%, preferably from 25 to 40%, based on the total mass of the core ingredients.

A further essential ingredient of the core is the vinylpyrrolidone-vinyl acetate copolymer. This copolymer is known per se and can be produced with any desired monomer mixing ratios. The commercially available Kollidon® VA64 (produced by BASF) which is preferably used is, for example, a 60:40 copolymer. It generally has a weight average molecular weight Mw, determined by light-scattering measurements, of about 45 000 to about 70 000. The amount of the vinylpyrrolidone-vinyl acetate copolymer in the core is 10 to 30%, preferably 15 to 25%, based on the total mass of the core ingredients.

Hydrophilic swellable polymers which are additionally present where appropriate in the core are, for example, hydroxypropylcellulose, hydroxypropylmethylcellulose, sodium carboxymethylcellulose, sodium carboxymethyl starch, polyacrylic acids and salts thereof.

Osmotically active additives which are additionally present where appropriate in the core are, for example, all water-soluble substances acceptable for use in pharmacy, such as, for example, the water-soluble excipients mentioned in pharmacopoeias or in “Hager” and “Remington Pharmaceutical Science”. It is possible in particular to use water-soluble salts of inorganic or organic acids or nonionic organic substances with high solubility in water, such as, for example, carbohydrates, especially sugars, sugar alcohols or amino acids. For example, the osmotically active additives can be selected from inorganic salts such as chlorides, sulphates, carbonates and bicarbonates of alkali metals or alkaline earth metals, such as lithium, sodium, potassium, magnesium, calcium, and phosphates, hydrogen phosphates or dihydrogen phosphates, acetates, succinates, benzoates, citrates or ascorbates thereof. It is furthermore possible to use pentoses such as arabinose, ribose or xylose, hexoses such as glucose, fructose, galactose or mannose, disaccharides such as sucrose, maltose or lactose or trisaccharides such as raffinose. The water-soluble amino acids include glycine, leucine, alanine or methionine. Sodium chloride is particularly preferably used according to the invention. The osmotically active additives are preferably present in an amount of from 10 to 30% based on the total mass of the core ingredients.

Pharmaceutically usual excipients which are additionally present where appropriate in the core are, for example, buffer substances such as sodium bicarbonate, binders such as hydroxypropylcellulose, hydroxypropylmethylcellulose and/or polyvinylpyrrolidone, lubricants such as magnesium stearate, wetting agents such as sodium lauryl sulphate and/or flow regulators such as colloidal silicon dioxide.

The present invention further relates to a process for producing an osmotic single-chamber system of the invention, where the components of the core are mixed together, subjected where appropriate to wet or dry granulation, and subsequently tabletted, and the core produced in this way is coated with the shell which is then covered where appropriate with a photoprotective and/or coloured coating, and which is provided with one or more orifices.

In a preferred embodiment of the present invention, the core components are subjected to a wet granulation during the production of the osmotic single-chamber system, because this process step improves the wettability of the ingredients of the tablet core, resulting in better penetration of the core by the entering gastrointestinal fluid, which frequently leads to faster and more complete release of the active ingredient.

In the osmotic two-chamber system, the core consists of two layers, one active ingredient layer and one osmosis layer. An osmotic two-chamber system of this type is described in detail for example in DE 34 17 113 C 2, the disclosure of which is incorporated herein by reference.

The active ingredient layer preferably comprises:
1 to 40% active ingredient (I),
50 to 95% of one or more osmotically active polymers, preferably polyethylene oxide of medium viscosity (40 to 100 mPa·s; 5% strength aqueous solution, 25° C.; preferably measured using a suitable Brookfield viscometer and a suitable spindle at a suitable speed of rotation, in particular using an RVT model Brookfield viscometer and a No. 1 spindle at a speed of rotation of 50 rpm or using a comparable model under corresponding conditions (spindle, speed of rotation)).

The osmosis layer preferably comprises:
40 to 90% of one or more osmotically active polymers, preferably polyethylene oxide of high viscosity (5000 to 8000 mPa·s; 1% strength aqueous solution, 25° C.; preferably measured using a suitable Brookfield viscometer and a suitable spindle at a suitable speed of rotation, in particular using an RVF model Brookfield viscometer and a No. 2 spindle at a speed of rotation of 2 rpm or using a comparable model under corresponding conditions (spindle, speed of rotation)).
10 to 40% of an osmotically active additive,
   where the difference from 100% in the individual layers is formed in each case independently of one another by one or more additional ingredients in the form of pharmaceutically usual excipients. The % data are in each case based on the total mass of the particular core layer.

The osmotically active additives used in the core of the osmotic two-chamber system may be the same as in the case of the single-chamber system described above. Sodium chloride is preferred in this connection.

The pharmaceutically usual excipients used in the core of the osmotic two-chamber system may be the same as in the case of the single-chamber system described above. Preference is given in this connection to binders such as hydroxypropylcellulose, hydroxypropylmethylcellulose and/or polyvinylpyrrolidone, lubricants such as magnesium stearate, wetting agents such as sodium lauryl sulphate and/or flow regulators such as colloidal silicon dioxide, and a colouring pigment such as iron oxide in one of the two layers to differentiate active ingredient layer and osmosis layer.

The present invention further relates to a process for producing the osmotic two-chamber system according to the invention, where the components of the active ingredient layer are mixed and granulated, the components of the osmosis layer are mixed and granulated, and then the two granules are compressed to a bilayer tablet in a bilayer tablet press. The core produced in this way is then coated with a shell, and the shell is provided with one or more orifices on the active ingredient side and subsequently also covered where appropriate with a coating.

In a preferred embodiment of the present invention, both the components of the active ingredient layer and the components of the osmosis layer are each subjected to dry granulation, in particular by means of roller granulation, in the production of the osmotic two-chamber system.

Preference is given according to the invention, because of the physicochemical properties of the active ingredient (I), to osmotic two-chamber systems (push-pull systems) in which the active ingredient layer and osmosis layer are separated, by way of example and advantageously formulated as 2-layer tablet. The advantages over osmotic single-chamber systems are in this case that the release rate is more uniform over a longer period, and that it is possible to reduce the system-related need for an excess of active ingredient.

The present invention further relates to medicaments comprising a solid, modified-release pharmaceutical dosage form according to the invention which can be administered orally and comprises the active ingredient (I).

The present invention further relates to the use of the solid, modified-release pharmaceutical dosage form according to the invention which can be administered orally and comprises the active ingredient (I) for the prophylaxis, secondary prophylaxis and/or treatment of disorders, in particular of arterial and/or venous thromboembolic disorders such as myocardial infarction, angina pectoris (including unstable angina), reocclusions and restenoses following an angioplasty or aortocoronary bypass, stroke, transient ischaemic attacks, peripheral arterial occlusive diseases, pulmonary embolisms or deep vein thromboses.

The present invention further relates to the use of the solid, modified-release pharmaceutical dosage form according to the invention which can be administered orally and comprises the active ingredient (I) for producing a medicament for the prophylaxis, secondary prophylaxis and/or treatment of disorders, in particular of arterial and/or venous thromboembolic disorders such as myocardial infarction, angina pectoris (including unstable angina), reocclusions and restenoses following an angioplasty or aortocoronary bypass, stroke, transient ischaemic attacks, peripheral arterial occlusive diseases, pulmonary embolisms or deep vein thromboses.

The present invention further relates to the use of 5-chloro-N-({(5S)-2-oxo-3-[4-(3-oxo-4-morpholinyl) phenyl]-1,3-oxazolidin-5-yl}methyl)-2-thiophenecarboxamide (I) for producing a solid, modified-release pharmaceutical dosage form according to the invention.

The present invention further relates to a method for the prophylaxis, secondary prophylaxis and/or treatment of arterial and/or venous thromboembolic disorders through administration of a solid, modified-release pharmaceutical dosage form according to the invention which can be administered orally and comprises the active ingredient (I).

The invention is explained in more detail below by preferred exemplary embodiments but is not restricted thereto. Unless indicated otherwise, all quantitative data below are based on percentages by weight.

Experimental Section

Unless indicated otherwise, the in vitro release investigations described below were carried out by the USP release method with apparatus 2 (paddle). The speed of rotation of the stirrer is 75 rpm (revolutions per minute) in 900 ml of a buffer solution of pH 6.8, which was prepared from 1.25 ml of ortho-phosphoric acid, 4.75 g of citric acid monohydrate and 27.46 g of disodium hydrogen phosphate dehydrate in 10 l of water. Also added to the solution where appropriate is ≦1% surfactant, preferably sodium lauryl sulphate. Tablet formulations are preferably released from a sinker as specified in the Japanese pharmacopoeia.

1. Tablet Formulations Based On Erosion Matrix Systems

1.1 Erosion Matrix Tablets Comprising Crystalline Active Ingredient (I)

Exemplary formulation 1.1.1
Tablet composition in mg/tablet

| | |
|---|---|
| Active ingredient (I), micronized | 25.0 mg |
| Microcrystalline cellulose | 10.0 mg |
| Lactose monohydrate | 26.9 mg |
| Hydroxypropylcellulose, type HPC-L (Nisso) | 52.0 mg |
| Hydroxypropylcellulose, type HPC-M (Nisso) | 10.0 mg |
| Sodium lauryl sulphate | 0.5 mg |
| Magnesium stearate | 0.6 mg |
| Hydroxypropylmethylcellulose, 15 cp | 1.8 mg |
| Polyethylene glycol 3350 | 0.6 mg |
| Titanium dioxide | 0.6 mg |
| | 128.0 mg |

Production:

A portion of the type L hydroxypropylcellulose and sodium lauryl sulphate are dissolved in water. The micronized active ingredient (I) is suspended in this solution. The suspension prepared in this way is sprayed as granulation liquid onto microcrystalline cellulose, HPC-L and HPC-M and lactose monohydrate in a fluidized bed granulation. Drying and sieving (0.8 mm mesh width) of the resulting granules is followed by addition of magnesium stearate and mixing. The mixture ready for compression obtained in this way is compressed to tablets with a diameter of 7 mm and a resistance to crushing of from 50 to 100 N. The tablets are subsequently coated with titanium dioxide which is suspended in an aqueous solution of hydroxypropylmethylcellulose (15 cp) and polyethylene glycol.

Exemplary formulation 1.1.2
Tablet composition in mg/tablet

| | |
|---|---|
| Active ingredient (I), micronized | 25.0 mg |
| Microcrystalline cellulose | 10.0 mg |
| Lactose monohydrate | 26.9 mg |
| Hydroxypropylcellulose, type HPC-L (Nisso) | 12.0 mg |
| Hydroxypropylcellulose, type HPC-M (Nisso) | 50.0 mg |
| Sodium lauryl sulphate | 0.5 mg |
| Magnesium stearate | 0.6 mg |
| Hydroxypropylmethylcellulose, 15 cp | 1.8 mg |
| Polyethylene glycol 3350 | 0.6 mg |
| Titanium dioxide | 0.6 mg |
| | 128.0 mg |

Production takes place in analogy to exemplary formulation 1.1.1

In vitro release from exemplary formulations 1.1.1 and 1.1.2:

| | | Time [min] | | | | |
|---|---|---|---|---|---|---|
| | | 120 | 240 | 480 | 720 | 960 |
| Release [%] | 1.1.1 | 38 | 74 | 94 | 96 | 97 |
| | 1.1.2 | 14 | 32 | 66 | 89 | 98 |

Method: USP paddle, 75 rpm, 900 ml of phosphate buffer of pH 6.8+0.5% sodium lauryl sulphate, JP sinker

1.2 Erosion Matrix Tablet Comprising Amorphous Active Ingredient (I)

Exemplary formulation 1.2
Tablet composition in mg/tablet
Melt extrudate:

| | |
|---|---|
| Active ingredient (I), micronized | 30.0 mg |
| Hydroxypropylcellulose, type HPC-M (Nisso) | 210.0 mg |
| Xylitol | 60.0 mg |
| | 300.0 mg |

| Tablets: | A | B | C |
|---|---|---|---|
| Melt extrudate, ground | 300.0 mg | 300.0 mg | 300.0 mg |
| Mannitol (Pearlitol, Roquette) | 195.0 mg | 100.0 mg | — |
| Hydroxypropylcellulose (type | — | — | 95.0 mg |
| HPC-L, Nisso) | | | |
| Hydroxypropylmethylcellulose | — | 95.0 mg | — |
| (15 cp) | | | |
| Microcrystalline cellulose | 50.0 mg | — | — |
| Colloidal silicon dioxide | 2.5 mg | 2.5 mg | 2.5 mg |
| (Aerosil 200, Degussa) | | | |
| Magnesium stearate | 2.5 mg | 2.5 mg | 2.5 mg |
| | 550.0 mg | 500.0 mg | 400.0 mg |

Production:

Micronized active ingredient (I), hydroxypropylcellulose and xylitol are mixed and processed in a twin screw extruder (Leistritz Micro 18 PH) with a die diameter of 2 mm. The mixture is extruded at a temperature of 195° C. (measured at the die outlet). The resulting extrudate strand is cut into pieces 1 to 2 mm in size and then ground in an impact mill.

After sieving (0.63 mm), the further excipients (see Table above) are mixed in with the ground extrudate, and this mixture is compressed to tablets with the oblong format of 15×7 mm (A+B) or 14×7 mm (C).

In vitro release from formulations 1.2 A to C:

Time [min]

| | | 240 | 480 | 720 | 1440 |
|---|---|---|---|---|---|
| Release [%] | A | 30 | 63 | 83 | 95 |
| | B | 27 | 56 | 77 | 99 |
| | C | 23 | 45 | 64 | 98 |

Method: USP paddle, 75 rpm, 900 ml of phosphate buffer of pH 6.8, JP sinker

A conventional fast-release tablet containing the same active ingredient amount of 30 mg of active ingredient (I) per tablet in micronized crystalline form achieves only incomplete release of active ingredient under the same conditions: in this case a plateau with only about 33% release of active ingredient is reached after 4 to 6 hours. By comparison therewith, the virtually complete release of active ingredient from the extrudate formulations A-C in the surfactant-free release medium shows a very marked increase in the solubility of the active ingredient (I). It was possible to achieve this by converting the active ingredient (I) into the amorphous state by melt extrusion processes.

2. Multiparticulate Preparations

2.1 Mini Tablets Comprising Crystalline Active Ingredient (I)

Exemplary formulation 2.1
Tablet composition in mg/mini tablet

| | |
|---|---|
| Active ingredient (I), micronized | 0.50 mg |
| Hydroxypropylcellulose (Klucel HXF, Hercules) | 5.91 mg |
| Hydroxypropylcellulose (type HPC-L, Nisso) | 0.04 mg |
| Sodium lauryl sulphate | 0.01 mg |
| Magnesium stearate | 0.04 mg |
| | 6.50 mg |

Production:

Klucel HXF hydroxypropylcellulose is granulated with an aqueous suspension of active ingredient (I) and HPC-L type hydroxypropylcellulose and sodium lauryl sulphate. Drying and sieving of the resulting granules are followed by addition of magnesium stearate and mixing. The mixture ready for compression obtained in this way is compressed to 2 mm mini tablets of 6.5 mg. The release from an amount of the mini tablets (50) equivalent to 25 mg of active ingredient (I) is detailed below:

In vitro release from formulation 2.1:

Time [min]

| | 240 | 480 | 720 | 1200 |
|---|---|---|---|---|
| Release [%] | 14 | 31 | 52 | 89 |

Method: USP paddle, 75 rpm, 900 ml of phosphate buffer of pH 6.8+0.5% sodium lauryl sulphate

2.2 Pellets Comprising Amorphous Active Ingredient (I)

Exemplary formulation 2.2.1
Composition in mg of active ingredient (I) per 30 mg single dose
Melt extrudate

| | |
|---|---|
| Active ingredient (I), micronized | 30.0 mg |
| Hydroxypropylcellulose, type Klucel HXF (Hercules) | 510.0 mg |
| Xylitol | 60.0 mg |
| | 600.0 mg |

Shell coating

| | |
|---|---|
| Hydroxypropylmethylcellulose, 3 cp | 15.0 mg |
| Magnesium stearate | 6.9 mg |
| Poly(ethyl acrylate, methyl methacrylate) 30% dispersion | 126.0 mg* |
| (Eudragit NE 30 D, Röhm/Degussa) | |
| Polysorbate 20 | 0.3 mg |
| | 60.0 mg** |

*equivalent to 37.8 mg of coating dry matter
**coating dry matter

Production:

Micronized active ingredient (I), hydroxypropylcellulose and xylitol are mixed. 1.5 kg of this mixture are processed in a twin screw extruder (Leistritz Micro 18 PH) with a die diameter of 2 mm. The mixture is extruded at a temperature of 200° C. (measured at the die outlet). The resulting extrudate strand is cut into pieces 1.5 mm in size. After sieving to remove the fines, the pellets are coated in a fluidized bed. For this purpose, an aqueous coating dispersion consisting of the components described above and 20% solids content is sprayed onto the particles. After drying and sieving, the pellets can be packed for example into glass bottles, sachets or hard gelatin capsules.

Exemplary formulation 2.2.2
Composition in mg of active ingredient (I) per 30 mg single dose
Melt extrudate

| | |
|---|---|
| Active ingredient (I), micronized | 30.0 mg |
| Hydroxypropylcellulose, type Klucel HXF (Hercules) | 570.0 mg |
| | 600.0 mg |

Shell coating

| | |
|---|---|
| Hydroxypropylmethylcellulose, 3 cp | 15.0 mg |
| Magnesium stearate | 6.9 mg |
| Poly(ethyl acrylate, methyl methacrylate) 30% dispersion | 126.0 mg* |
| (Eudragit NE 30 D, Röhm/Degussa) | |
| Polysorbate 20 | 0.3 mg |
| | 60.0 mg** |

*equivalent to 37.8 mg of coating dry matter
**coating dry matter

Production: analogous to 2.2.1

Although a similar procedure/process for producing multiparticulate slow release preparations is described in EP 1 113 787, the difference is that in Examples 2.2.1 and 2.2.2 described herein the active ingredient (I) is converted into the amorphous form because of suitable process parameters. An increase in the solubility of active ingredient in particular is achieved thereby:

In vitro release from formulations 2.2.1 and 2.2.2

Time [min]

| | | 240 | 480 | 720 | 1440 |
|---|---|---|---|---|---|
| Release [%] | 3.2.1 | 34 | 69 | 91 | 95 |
| | 3.2.2 | 30 | 57 | 80 | 94 |

Method: USP paddle, 75 rpm, 900 ml of phosphate buffer of pH 6.8

Dosage forms comprising the active ingredient (I) in crystalline form achieve a release of only about 33% under the same conditions (see also the discussion of the release results for exemplary formulation 1.2)

3. Osmotic Systems

3.1 Single-chamber System Comprising Crystalline Active Ingredient (I)

Exemplary formulation 3.1
Tablet composition in mg/tablet (declared content = 30 mg/tablet)
Core

| | |
|---|---|
| Active ingredient (I), micronized | 36.0 mg |
| Xanthan gum (Rhodigel TSC, Rhodia) | 100.0 mg |
| Copolyvidone (Kollidon VA 64, BASF) | 55.0 mg |
| Sodium chloride | 55.0 mg |
| Sodium bicarbonate | 17.5 mg |
| Sodium carboxymethyl starch | 23.0 mg |
| Hydroxypropylmethylcellulose (5 cp) | 10.0 mg |
| Sodium lauryl sulphate | 0.5 mg |
| Colloidal silicon dioxide (Aerosil 200, Degussa) | 1.5 mg |
| Magnesium stearate | 1.5 mg |
| | 300.0 mg |

Shell (osmotic membrane)

| | |
|---|---|
| Cellulose acetate | 19.95 mg |
| Polyethylene glycol 400 | 1.05 mg |
| | 21.00 mg |

Production:

Xanthan gum, copolyvidone, sodium chloride, sodium bicarbonate and sodium carboxymethylcellulose are mixed and then subjected to wet granulation with an aqueous suspension of active ingredient (I) and hydroxypropylmethylcellulose. Drying and sieving are followed by admixture of Aerosil and magnesium stearate, and the mixture ready for compression obtained in this way is compressed to tablets with a diameter of 8 mm. The tablet cores are coated with acetone solution of cellulose acetate and polyethylene glycol and dried. Subsequently, two orifices each 1 mm in diameter are made in each tablet using a hand drill.

In vitro release from exemplary formulation 3.1

Time [min]

| | 240 | 480 | 720 | 1440 |
|---|---|---|---|---|
| Release [%] | 21 | 54 | 72 | 90 |

Method: USP paddle, 100 rpm, 900 ml of phosphate buffer of pH 6.8+1.0% sodium lauryl sulphate, JP sinker

3.2 Two-chamber System Comprising Crystalline Active Ingredient (I)

Exemplary formulation 3.2
Tablet composition in mg/tablet (declared content = 30 mg/tablet)
Core
Active ingredient layer

| | |
|---|---|
| Active ingredient (I), micronized | 33.0 mg |
| Hydroxypropylmethylcellulose (5 cp) | 8.2 mg |
| Polyethylene oxide* | 122.2 mg |
| Colloidal silicon dioxide (Aerosil 200, Degussa) | 1.3 mg |
| Magnesium stearate | 0.8 mg |
| | 165.5 mg |

Osmosis layer

| | |
|---|---|
| Hydroxypropylmethylcellulose (5 cp) | 4.1 mg |
| Sodium chloride | 23.9 mg |
| Polyethylene oxide** | 52.9 mg |
| Red iron oxide | 0.8 mg |
| Magnesium stearate | 0.2 mg |
| | 81.9 mg |

Shell (osmotic membrane)

| | |
|---|---|
| Cellulose acetate | 29.07 mg |
| Polyethylene glycol 400 | 1.53 mg |
| | 30.60 mg |

*Viscosity of 5% strength aqueous solution (25° C., RVT model Brookfield viscometer, No. 1 spindle, speed of rotation: 50 rpm): 40-100 mPa · s (e.g. POLYOX ™ Water-Soluble Resin NF WSR N-80; Dow)
**Viscosity of 1% strength aqueous solution (25° C., RVF model Brookfield viscometer, No. 2 spindle, speed of rotation: 2 rpm): 5000-8000 mPa · s (e.g. POLYOX ™ Water-Soluble Resin NE WSR Coagulant; Dow)

Production:

The components of the active ingredient layer are mixed and subjected to dry granulation (roller granulation). The components of the osmosis layer are likewise mixed and subjected to dry granulation (roller granulation). The two granules are compressed in a bilayer tablet press to a bilayer tablet (diameter 8.7 mm). The tablets are coated with an acetone solution of cellulose acetate and polyethylene glycol and dried. An orifice 0.9 mm in diameter is then made on the active ingredient side of each tablet using a hand drill.

In vitro release from exemplary formulation 3.2

Time [min]

| | 240 | 480 | 720 | 1200 |
|---|---|---|---|---|
| Release [%] | 21 | 54 | 81 | 99 |

Method: USP paddle, 100 rpm, 900 ml of phosphate buffer of pH 6.8+1.0% sodium lauryl sulphate, JP sinker

1. A solid, modified-release pharmaceutical dosage form which can be administered orally and comprises 5-chloro-N-({(5S)-2-oxo-3-[4-(3-oxo-4-morpholinyl)phenyl]-1,3-oxazolidin-5-yl}methyl)-2-thiophenecarboxamide (I), characterized in that 80% of the active ingredient (I) are released in a period of from 2 to 24 hours in the USP release method with apparatus 2 (paddle).

2. The pharmaceutical dosage form according to claim 1, characterized in that 80% of the active ingredient (I) are released in a period of from 4 to 20 hours in the USP release method with apparatus 2 (paddle).

3. The pharmaceutical dosage form according to claim 1, characterized in that the active ingredient (I) is present in crystalline form.

4. The pharmaceutical dosage form according to claim 3, comprising the active ingredient (I) in micronized form.

5. The pharmaceutical dosage form according to claim 4, comprising the active ingredient (I) in hydrophylized form.

6. The pharmaceutical dosage form according to claim 1, characterized in that the active ingredient (I) is present in amorphous form.

7. The pharmaceutical dosage form according to claim 6, characterized in that the active ingredient (I) has been amorphized by melt extrusion.

8. The pharmaceutical dosage form according to claim 7, characterized in that the polymer employed in the melt extrusion is hydroxypropylcellulose (HPC) or polyvinylpyrrolidone (PVP), the proportion of polymer in the melt extrudate is at least 50%, and the active ingredient (I) is present in the melt extrudate in a concentration of from 1 to 20%.

9. The pharmaceutical dosage form according to claim 7, characterized in that at least one pharmaceutically suitable substance is added in a concentration of from 2 to 40% as plasticizer and/or to depress the melting point of the active ingredient (I).

10. The pharmaceutical dosage form according to claim 9, characterized in that the pharmaceutically suitable additive is a sugar alcohol.

11. The pharmaceutical dosage form according to claim 1, wherein said dosage form is based on an erosion matrix system.

12. The pharmaceutical dosage form according to claim 11, characterized in that the active ingredient (I) is present in amorphous form.

13. The pharmaceutical dosage form according to claim 1, comprising hydroxypropylcellulose or hydroxypropylmethylcellulose or mixtures of hydroxypropylcellulose and hydroxypropylmethylcellulose as hydrophilic matrix former.

14. The pharmaceutical dosage form according to claim 11, characterized in that the active ingredient (I) is present in a concentration of between 1 and 50%.

15. A process for producing a pharmaceutical dosage form according to claim 11, comprising producing an extrudate comprising the active ingredient (I) by melt extrusion, grinding and mixing it with further tabletting excipients and then compressing it to tablets by direct tabletting.

16. A multiparticulate pharmaceutical dosage form according to claim 1.

17. The multiparticulate pharmaceutical dosage form according to claim 16, characterized in that the active ingredient (I) is present in amorphous form.

18. The multiparticulate pharmaceutical dosage form according to claim 16, comprising hydroxypropylcellulose as hydrophilic matrix former.

19. The multiparticulate pharmaceutical dosage form according to claim 18, characterized in that hydroxypropylcellulose is present as hydrophilic matrix former in a concentration of between 10 and 99%.

20. The multiparticulate pharmaceutical dosage form according to claim 16, characterized in that the active ingredient (I) is present in a concentration of between 1 and 30%.

21. The multiparticulate pharmaceutical dosage form according to claim 16, characterized in that the diameter of the particles is between 0.5 and 3.0 mm.

22. The multiparticulate pharmaceutical dosage form according to claim 21, characterized in that the diameter of the particles is between 1.0 and 2.5 mm.

23. A pharmaceutical dosage form comprising multiparticulate pharmaceutical dosage forms according to claim 16.

24. The pharmaceutical dosage form according to claim 23 in the form of a capsule, of a sachet or of a tablet.

25. A process for producing a multiparticulate pharmaceutical dosage form as defined in claim 16, comprising producing by melt extrusion an extrudate strand comprising the active ingredient (I) and cutting said strand.

26. The process according to claim 25, further comprising roundings the articles obtained after cutting the extrudate strand.

27. The process according to claim 25, further comprising coating said articles.

28. The pharmaceutical dosage form according to claim 1, wherein said dosage form is based on an osmotic release system.

29. The pharmaceutical dosage form according to claim 28, characterized in that the active ingredient (I) is present in amorphous form.

30. The pharmaceutical dosage form according to claim 28, consisting of an osmotic single-chamber system comprising a core comprising
2 to 30% active ingredient (I)
20 to 50% xanthan,
10 to 30% of a vinylpyrrolidone-vinyl acetate copolymer,
and a shell consisting of a water-permeable material which is impermeable for the components of the core and has at least one orifice.

31. The pharmaceutical dosage form according to claim 30, additionally comprising sodium chloride as osmotically active additive in the core.

32. The pharmaceutical dosage form according to claim 30, characterized in that the shell consists of cellulose acetate or of a mixture of cellulose acetate and polyethylene glycol.

33. A process for producing an osmotic single-chamber system as defined in claim 30, comprising mixing together the components of the core, granulating and tabletting them, coating the core produced in this way with a shell, and finally providing the shell with one or more orifices.

34. The pharmaceutical dosage form according to claim 28, consisting of an osmotic two-chamber system comprising a core having an active ingredient layer comprising
1 to 40% active ingredient (I),
50 to 95% of one or more osmotically active polymers,
and an osmosis layer comprising
40 to 90% of one or more osmotically active polymers,
10 to 40% of an osmotically active addition,
and a shell consisting of a water-permeable material which is impermeable for the components of the core and has at least one orifice.

35. The pharmaceutical dosage form according to claim 34, which comprises polyethylene oxide having a viscosity of from 40 to 100 mPa·s (5% strength aqueous solution, 25° C.) as osmotically active polymer in the active ingredient layer in the core, and comprises polyethylene oxide having a viscosity of from 5000 to 8000 mPa·s (1% strength aqueous solution, 25° C.) as osmotically active polymer in the osmosis layer in the core.

36. The pharmaceutical dosage form according to claim 34, characterized in that the shell consists of cellulose acetate or of a mixture of cellulose acetate and polyethylene glycol.

37. A process for producing an osmotic two-chamber system as defined in claim 34, comprising
mixing and granulating the components of the active ingredient layer and
mixing and granulating the components of the osmosis layer,
subsequently compressing the two granules in a bilayer tablet press to a bilayer tablet.
coating the core produced in this way with the shell, and
providing the shell with one or more orifices on the active ingredient side.

38. A medicament comprising a solid pharmaceutical dosage form which can be administered orally and has a modified release, as defined in claim 1, of the active ingredient (I).

39. A method for the prophylaxis, secondary prophylaxis and/or treatment of a thromboembolic disorder, comprising administering to a patient a therapeutically effective amount of the pharmaceutical dosage form of claim 1.

40. The method of claim 39, wherein the thromboembolic disorder is myocardial infarction, angina pectoris, reocclusion and restenosis following an angioplasty or aortocoronary bypass, stroke, transient ischaemic attack, peripheral arterial occlusive disease, pulmonary embolism or deep vein thrombosis.
